(19) United States
(12) Patent Application Publication
(10) Pub. No.: US 2005/0208158 A1
(43) Pub. Date: Sep. 22, 2005

(54) Pharmacological composition based on biologically active substances obtained from tribulus terrestris l

(76) Inventor: Blagoy Petrov Alexiev, Sofia (BG)

Correspondence Address: AKERMAN SENTERFITT, P.O. BOX 3188, WEST PALM BEACH, FL, 33402-3188, US

(21) Appl. No.: 10/505,800

(22) PCT Filed: Sep. 19, 2002
(86) PCT No.: PCT/BG02/00024

§ 371 Date: Aug. 25, 2004

(30) Foreign Application Priority Data
Feb. 25, 2002 (BG) 106435

(51) Int. Cl. A61K 35/78

(52) U.S. Cl. 424/757

(57) ABSTRACT

The invention relates to a pharmacological composition based on biologically active substances obtained from leaves, stems and fruit of the plant Tribulus terrestris L. to be used as an agent strengthening the immune system, increasing T-cells and anti-bodies number and testosterone quantity in human organism and having a strong anti-virus and anti-inflammatory effect in the form of a medicine or as an addition to food. The pharmacological composition represents a combination of bio-active zinc, bio-active selenium and a complex of steroid saponins obtained from leaves, stems and fruit of the plant Tribulus terrestris l. and consisting of furostanols spirostanols, sapogenins, sterols, flavonoids and other biologically active substances typical of this plant.

### FIELD OF THE INVENTION

The invention relates to a pharmacological composition obtained on the basis of biologically active substances from leaves, stems and fruit of the plant Tribulus terrestris L. to be used as an agent strengthening the immune system, increasing T-cells and antibodies number and testosterone hormone level in the organism as well as an agent of a strong anti-virus and anti-inflammatory effect which may be used both as a medicine and as an addition to food.

### PRIOR ART

It is known that zinc is used in different forms for improving sexual activity in combination with other active substances such as an extract of plant Saw Palmetto (pumpkin seeds), and that selenium is also used in different forms as an agent helping to strengthen the immune system of the organism.

A prophylactic agent is known in the form of solid gelatin capsules represent rig a homogeneous mixture of dried and ground bulbs of garlic, dry extract of ginseng, a mixture of vitamins A, C, E and the microelements iron, manganese and zinc used for strengthening the immune system and improving the protective forces of the organism prophylactically against infectious diseases. /BG 100802/

An agent is known for stimulation of the sexual activity representing a complex of steroid saponins of furostanol and spirostanol type obtained from leaves, stems and fruit of the plant Tribulus Terrestris L. (BG 27584)

A disadvantage of the latter is that it has been synthesized on the basis of furostanols and spirostanols as a result of which it has an effect only on the sexual activity of the organism.

A method is known of isolation of a standard mixture of steroid saponins mainly of furostanol type from Tribulus Terrestris L. with a view to obtaining a tribestan-substance as the bio-active material of an agent used for stimulating the sexual activity. (BG 52085)

A disadvantage of the tribestan -substance obtained according to this method is the high content of furostanol steroid saponins and the low content of other substances having a biological action which result in the achievement of a given pharmacological effect connected with the sexual activity only, but it has no complex effect on the other systems and functions of the human organism.

### SUMMARY OF THE INVENTION

The purpose of the invention is to create an anti-virus complex of an anti-inflammatory and sexual activity stimulating effect, for strengthening the immune protection by means of the general action of the complex of bio-active substances of mineral and vegetal origin on the whole organism.

The basis of the invention consists in creating a pharmacological composition based on biologically active substances obtained from the plant Tribulus Terrestris L. and being a combination of bio-active zinc and bio-active selenium of high biological capacity and a complex of natural substances of biological action representing steroid saponins obtained from the plant Tribulus terrestris l. and consisting of furostanols spirostanols, sapogenins, sterols, flavonoids and other biologically active substances typical of this plant.

The complex of steroid saponins contains furostanols, spirostanols sapogenins, sterols, flavonoids and other biologically active substances typical of this plant in the following ratios in % by weight: furostanols from 1 to 90%, spirostanols from 1 to 75%, sapogenins, from 0.01 to 80% sterols from 0.01% to 23%, flavonoids and other bio-active substances from 0.0001 to 24%.

The composition contains a complex of steroid saponins obtained from the plant Tribulus Terrestris L., bio-active zinc and bio-active selenium included in a pharmaceutical or a nutritive base in the following ratios in % by weight: a complex of steroid saponins obtained from Tribulus Terrestris L. from 1 to 40%, bio-active zinc from 0.001 to 10% , bio-active selenium from 0.001 to 2% and a pharmaceutical or a nutritive base from 1 to 55%.

The pharmaceutical or the nutritive base contains the following components in ratios in % by weight: micro-cristalline cellulose from 1 to 50%, dicalcium phosphate from1 to 35%, stearic acid from 0 to 10%, magnesium stearate from 01. to 10%. hidroxypropylic cellulose from 0 to 5% and highly dispersed silicon dioxide from 0.1 to 3%.

The pharmacological composition and especially the complex of steroid saponins obtained from Tribulus Terrestris L. contained in as a basic biologically active component of vegetal origin are made by additionally processing at negative temperatures the drug obtained in advance or the extract obtained from the plant Tribulus terrestris L. until obtaining a precise concentration of the biologically active substances in the complex of steroid saponins.

The pharmacological composition, subject of the invention is used for treatment or prophylaxis in case of diseases caused by viruses and especially HIV, herpes simplex virus 1, herpes simplex virus 2 and viruses causing flu and cold.

The pharmacological composition, subject of the invention is used for treatment or prophylaxis in case of inflammation of varicose veins, capillaries, kidneys, bladder, prostate gland and as an agent of marked anti-inflammatory effect.

The pharmacological composition is used to increase testosterone level, sexual activity, spermatogenesis and ovogenesis.

The pharmacological composition is used to strengthen the immune system and improve the resistant forces of the organism by increasing anti-bodies and T-cells number in human organism.

The pharmacological composition is used for treatment or prophylaxis as a solid medicine to taken orally or as an addition to food.

The Advantages of the Invention are as Follows:

The pharmacological composition, subject of the invention, contains the full complex of natural substances of biological action contained in the plant of Tribulus terrestris L. and combined with zinc and selenium of high biological capacity achieved as a result of the use of a special technology for their production guaranteeing the high biological capacity without an additional chemical processing of the bio-active substances of vegetal and mineral origin and of their composition being further necessary.

Drug processing or additional extract of leaves, stems and fruit of the plant Tribulus terrestris L. at negative temperatures causes a considerable increase of the content of different chemical groups of natural substances in the complex of steroids saponins which, combined with bio-active zinc and bio-active selenium, have a strong effect on biological processes taking place in protective cells and when anti-bodies are formed in the organism, as well as a complex effect on the biological processes in all other cells of the organism on which the biologically active substances have an effect and which are formed as a result of the interaction between the components of the combination.

The pharmacological composition, subject of the invention, is characterized by the presence of precisely determined biologically active substances in precise ratios having a complex effect on the biological processes in the cells of a determined bio-chemical medium, resulting in a stimulation of the biological processes in the cells or a rejection or destruction of the harmful substances accumulated in case of disturbed metabolism and causing inflammations or reduction of the resistant forces of the organism thus having a simultaneous effect on different systems of human organism.

As a result of the complex of biologically active substances contained in the pharmacological composition, subject of the invention, being absorbed, the biological and chemical media of many cells in the organism change thus increasing the possibility of destroying the viruses having penetrated in to the organism by optimizing the bio-chemical medium of blood plasma.

It is possible to use the pharmacological composition, subject of the invention, as a medicine or an addition to food which have a rapid effect, no harmful side effects on the organism in case of prolonged or short-term use in clinical conditions or at home and high bio-activity.

### DETAILED DESCRIPTION

The following examples are illustrative of the invention but not limiting it: The pharmacological composition according to the present invention and especially the complex of steroid saponins of the same are obtained by the processing or extracting at negative temperatures the drug made of leaves, stems and fruit of the plant Tribulus terrestris L. collected in special conditions until obtaining an active component containing complex of steroid saponins in a precise ratio in % by weight, this active component being included as the basic vegetal active component in the solid medicine made to be taken orally or in the addition to food according to examples 1 to 4 as follows:

### EXAMPLE 1

Solid medicine to be taken orally including the pharmacological composition according to the invention, representing a tablet and containing the following components:

0.250 g vegetal active powder component obtained from leaves, stems and fruit of the plant Tribulus Terrestris L. containing a complex of steroid saponins in % by weight as follows:

furostanols from 1 to 38 to
spirostanols from 1 to 43%
sapogenins from 0.1 to 28%
sterols from 0.01 to 10%
flavonoids and other active substances from 0.0001 to 12%
Mineral active components:
bio-active zinc—0.01 g
bio-active selenium—0.000003 g
Medicinal base containing
monocrystalline cellulose—0.300 g
dicalcium phosphate—0.100 g
stearic acid—0.027 g
magnesium stearate—0.025 g
hydroxypropylic cellulose—0.020 g
highly dispersed silicon dioxide—0.001 g

### EXAMPLE 2

Solid medicine to be taken orally including the pharmacological composition according to the invention, representing a tablet and containing the following components:

0.250 g vegetal active component representing a total extract of leaves, stems and fruit of the plant Tribulus Terrestris L. and containing a complex of steroid saponins in % by weight as follows:

furostanols from 1 to 28%
spirostanols from 1 to 73%
sapogenins from 0.01 to 38%
sterols from 0.01 to 12%
flavonoids and other active substances from 0.0001 to 14%
Mineral active components:
bio-active zinc—0.010 g
bio-active selenium—0.000007 g
Medicinal base containing
Micro-crystalline cellulose—0.300 g
dicalcium phosphate—0.100 g
stearic acid—0.027 g
magnesium stearate—0.025 g
hydroxypropylic cellulose—0.020 g
highly dispersed silicon dioxide—0.001 g

### EXAMPLE 3

Addition to food including the pharmacological composition according to the invention, representing a hard gelatin capsule and containing the following components:

0.250 g vegetal active component representing a total extract of leaves, stems and fruit of the plant Tribulus Terrestris L. and containing a complex of steroid saponins in % by weight as follows:

0.250 g vegetal active powder component obtained from leaves, stems and fruit of the plant Tribulus Terrestris L. containing a complex of steroid saponins in % by weight as follows:

furostanols from 1 to 22%
spirostanols from 1 to 74%
sapogenins from 0.1 to 32%
sterols from 0.01 to 11%
flavonoids and other active substances from 0.0001 to 11%
Mineral active components:
bio-active zinc—0.01g
bio-active selenium—0.000002 g
Nutritive base containing
Micro-crystalline cellulose—0.350g
dicalcium phosphate—0.100 g
magnesium stearate—0.025 g
highly dispersed silicon dioxide—0.001 g

### EXAMPLE 4

Addition to food including the pharmacological composition according to the invention, representing a hard gelatin capsule and containing the following components:

0.250 g vegetal active component representing a total extract of leaves, stems and fruit of the plant Tribulus Terrestris L. and containing a complex of steroid saponins in % by weight s follows:

furostanols from 1 to 26%
spirostanols from 1 to 69%
sapogenins from 0.01 to 37%
sterols from 0.01 to 14%
flavonoids and other active substances from 0.0001 to 15%
Mineral active components:
bio-active zinc—0.01 g
bio-active selenium—0.000009 g
Medicinal or nutritive base containing
micro-crystalline cellulose—0.350 g
dicalcium phosphate—0.100 g
magnesium stearate—0.025 g
highly dispersed silicon dioxide—0.001 g

In another version of the present invention the pharmacological composition and especially the complex of steroid saponins for the same are made after extracting the drug gathered in special conditions from leave, stems and fruit of the plant Tribulus Terrestris l. and processing further the extract thus obtained at negative temperatures until obtaining an active component containing a complex of steroid saponins in a precise ratio in % by weight, this active component being included as the basic vegetal active component in the compositions made to be used as solid medicines or additions to food.

### USE OF THE INVENTION

The medicine described in example 1 has been clinically tested in Sofia, Bulgaria on patients infected with herpes simplex virus 1 and herpes simplex virus 2 (genitals herpes). It has been administered according to individual prescriptions depending on the symptoms and the degree of the decease and taken in doses of 2-4 tablets twice to six times per day. The dose has had no negative effect on the organism. Great tolerance and reliability have been observed and the following pharmacological effects have resulted:
a positive result of anti-virus action on herpes simplex virus 1 in 67% of the cases;
a positive result of anti-virus action on herpes simplex virus 2 in 86% of the cases;
a positive result of anti-inflammation varicose veins, capillaries, kidneys, bladder, prostate gland in 59% of the cases;
a reduction of exaggerated prostate gland in 89% of the cases.

The medicine described in example 2 has been clinically tested in Johannesburg, SAR, on patients infected with HIV. It has been administered according to individual prescriptions depending on the degree of lesion of the immune system and taken in doses 2-4 tablets twice to six times per day. Side effects have not been observed, and the following; pharmacological effects have been proved:
a positive result of anti-virus action on HIV in 92% of the cases;
an increase of anti-bodies and T-cells number by 89%;
a improvement of blood circulation in limbs capillaries and veins by 58%;

The addition to food described in example 3 has been clinically tested in Sofia, Bulgaria, during the treatment of flu and cold caused by viruses and of inflammation of varicose veins, capillaries, kidneys, bladder and prostate gland. It has been administered according to individual prescriptions depending on the seriousness and form of the disease. Side effects have not been observed at the end of the treatment:
a positive result in 91% of the patients having suffered from flu and cold;
a positive result in 64% of the patients suffering from inflammation of kidneys, bladder, prostate gland, varicose veins and capillaries.

The addition to food described in example 4 has been tested in Plovdiv, Bulgaria, during the treatment of patient suffering from sexual insufficiency. The following results have been observed at the end of the treatment:
increased sexual activity in 80% of the cases;
increase of sperm quantity and quality in 78% of the case;
reduction of exaggerate gland in 88% of the cases.

1. Pharmacological composition based on biologically active substances obtained from the plant Tribulus Terrestris L, characterized by that it is a combination of bio-active zinc, bio-active selenium and a complex of natural substances of biological action representing steroid saponins obtained from leaves, stems and fruit of the plant Tribulus terrestris l. and consisting of furostanols, spirostanols, sapogenins, sterols, flavonoids and other biologically active substances typical of this plant.

2. Pharmacological composition according to claim 1, characterized in that the complex of steroid saponins contains furostanols, spirostanols sapogenins, sterols, flavonoids and other biologically active substances typical of this plant in the following ratios in % by weight:
furostanols from 1 to 90%,
spirostanols from 1 to 75%,
sapogenins, from 0.01 to 80%,
sterols from 0.01% to 23%,
flavonoids and other bio-active substances from 0.0001 to 24%.

3. Pharmacological composition according to claim 1, characterized in that the complex of steroid saponins obtained from the leaves, stems and fruit of the plant Tribulus Terrestris L., bio-active zinc and bio-active selenium are all included in a pharmaceutical or nutritive base in the following ratios in % weight:
a complex of steroid saponins obtained from Tribulus Terrestris L. from 1 to 40%,
bio-active zinc from 0.001 to 2%,
pharmaceutical or a nutritive base from 1 to 55%.

4. Pharmacological composition according to claim 3, characterized in that the pharmaceutical or the nutritive base contains the following components in ratios in % by weight:
micro-cristalline cellulose from 1 to 50%,
dicalcium phosphate from 1 to 35%,
stearic acid from 0 to 10%,
magnesium stearate from 01. to 10%,
hydroxypropylic cellulose from 0 to 5%
highly dispersed silicon dioxide from 0.1 to 3%.

5. A method of obtaining the pharmacological composition according to claim 1 characterized in that the extract from leaves, stems and fruit of the plant Tribulus Terrestris L. is processed additionally at negative temperatures until obtaining a precise concentration of the biologically active substances contained in the complex of steroid saponins.

6. A method for treatment or prophylaxis of diseases caused by viruses and especially HIV, herpes simplex virus 1, herpes simplex virus 2 and viruses causing flu and cold comprising administering to a patient the pharmacological composition according to claim 1.

7. A method for treatment or prophylaxis in case of inflammation of at least one of varicose veins, capillaries, kidneys, bladder, and prostate gland and for providing a marked anti-inflammatory effect comprising administering to a patient the pharmacological composition according to claim 1.

8. A method for increasing good cholesterol concentration and reducing bad cholesterol concentration in blood comprising administering to a patient the pharmacological composition according to claim 1.

9. A method for strengthening the immune system and improving the resistant forces of human organism by increasing anti-bodies and T-cells number in human organism comprising administering to a patient the pharmacological composition according to claim 1.
